(19) United States
(12) Patent Application Publication
(10) Pub. No.: US 2002/0058037 A1
(43) Pub. Date: May 16, 2002

(54) Use of anti-gp-39 antibodies for treatment and/or reversal of lupus and lupus associated kidney disease

(75) Inventors: Randolph J. Noelle, Cornish, NH (US); Christopher M. Burns, Lyme, NH (US)

Correspondence Address: Pillsbury Winthrop LLP, Intellectual Property Group, East Tower, Ninth Floor, 1100 New York Avenue, N.W., Washington, DC, 20005-3918, US

(73) Assignee: Trustees of Dartmouth College, Hanover, NH (US)

(21) Appl. No.: 09/855,528

(22) Filed: May 16, 2001

Related U.S. Application Data
(63) Continuation of application No. 09/054,488, filed on Apr. 3, 1998, now abandoned.
Application No. 09/054,488 is a continuation-in-part of application No. 08/742,480, filed on Nov. 1, 1996.
Application No. 08/742,480 is a continuation of application No. 08/338,975, filed on Nov. 14, 1994.
Application No. 08/338,975 is a continuation of application No. 07/835,799, filed on Feb. 14, 1992, now abandoned.

(51) Int. Cl. A61K 39/395; A61K 38/19

(52) U.S. Cl. 424/146.1; 424/85.1

(57) ABSTRACT

A method of treating lupus using anti-gp39 antibodies or fragments is provided. Such treatment has been shown to reverse disease, and in particular lupus-associated kidney disease, the major killer of lupus subjects.

### CROSS-REFERENCE TO RELATED APPLICATIONS

This application is a continuation of U.S. Ser. No. 09/054,488, filed Apr. 3, 1998, which is a continuation-in-part of U.S. Ser. No. 08/742,480, filed Nov. 1, 1996, which, in turn, is a continuation of Ser. No. 08/338,975, filed Nov. 14, 1994, abandoned, in turn, a continuation of Ser. No. 07/835,799, filed Feb. 14, 1992, now abandoned.

### FIELD OF THE INVENTION

The present invention relates to a counter-receptor, referred to alternatively in the literature as CD40CR, gp39, or most recently CD154 for the CD40 B-cell antigen, and to soluble ligands for this receptor, including fusion molecules comprising at least a portion of CD40 protein. It is based, at least in part, on the discovery that a soluble CD40/immunoglobulin fusion protein was able to inhibit helper T-cell mediated B-cell activation by binding to a novel 39 kD protein receptor on helper T-cell membranes. The present invention provides for a substantially purified CD40CR receptor; for soluble ligands of CD40CR, including anti-gp39 antibodies and fragments thereof, as well as fusion molecules comprising at least a portion of CD40 protein; and for methods of controlling B-cell activation which may be especially useful in the treatment of allergy or autoimmune disease. More specifically, the present invention relates to the use of anti-gp39 antibodies for treating systemic lupus erythematosus (SLE) or drug induced lupus.

### BACKGROUND OF THE INVENTION

Studies by Mitchison, Benacerraf and Raff first suggested that physical interactions between Tₕ and B-cells were essential in the development of humoral immune responses. Later studies documented that Tₕ formed physical conjugates with class II major histocompatibility complex (MHC) compatible, antigen-presenting B-cells (Vitetta et al., Immunol. Rev., 99:193-239 (1987)) and that it was the B-cells within these conjugates that responded to Tₕ (Barrett et al., J. Immunol., 143:1745-1754 (1989)). With the discovery that Tₕ-derived lymphokines exerted potent growth and differentiative effects on B-cells, it was proposed that soluble factor(s) released in proximity by activated Tₕ mediated the activation of the interacting B-cell. However, none of the molecularly cloned lymphokines, alone or in combination, manifested the ability to induce B-cell cycle entry. Unlike soluble factors, plasma membrane fractions from activated Tₕ induced B-cell cycle entry (Hodgkin et al., J Immunol., 145:2025-2034 (1990); Noelle et al., J. Immunol., 146:1118-1124 (1991)). Studies using purified plasma membrane fractions from activated Th suggested that a protein expressed on the membrane of activated Th was responsible for initiating humoral immunity (Noelle et al., J. Immunol., 146:1118-1124 (1991); Bartlett et al., J. Immunol., 145:3956-3962 (1990)).

Purified plasma membranes from activated Tₕ (PM^{ACT}) have been used to investigate the nature of this effector function (Hodgkin et al., J. Immunol., 145:2025-2034 (1990); Noelle et al., J. Immunol., 146:1118-1124 (1991)). PM^{ACT} from activated Tₕ, but not resting Tₕ (PM^{REST}) expressed an activity that induced B-cell cycle entry in an antigen-nonspecific, class II-unrestricted manner. In addition, it was shown that the activity expressed by PM^{ACT} required 4-6 hours of activation, de novo RNA synthesis and was protein in nature (Bartlett et al., J. Immunol, 145:3956-3962 (1990)).

### SUMMARY OF THE INVENTION

The present invention relates to a counter-receptor, termed CD40CR, for the CD40 B-cell antigen, and to soluble ligands for this receptor, including fusion molecules comprising at least a portion of CD40 protein. It is based, at least in part, on the discovery that a soluble CD40/immunoglobulin fusion protein was able to inhibit helper T-cell mediated B-cell activation by binding to a novel 39 kD receptor protein (termed “CD40CR” for CD40 counter-receptor) on helper T-cell membranes, and on the discovery that a monoclonal antibody, termed MR1, directed toward this 39 kD receptor was able to inhibit helper T-cell mediated activation of B-cells.

The present invention provides for a substantially purified CD40CR receptor for soluble ligands of CD40CR, including antibodies, as well as fusion molecules comprising at least a portion of CD40 protein; and for methods of controlling B-cell activation.

In particular embodiments of the invention, B-cell activation in a subject may be inhibited by contacting helper T cells of the subject with therapeutically effective amounts of a soluble ligand or CD40CR. Such inhibition of B-cell activation may be especially useful in the treatment of allergy or autoimmune disease.

More specifically, the present invention provides a method of treating lupus in a subject in need of such treatment, e.g. a patient with ongoing systemic lupus erythematosus, or drug-induced lupus, even in the advanced stages of the disease process (wherein kidney damage is often observed) by the administration of a therapeutically effective amount of an anti-gp39 antibody, e.g. the anti-human gp39 antibodies or fragments thereof disclosed in commonly assigned U.S. Ser. No. 08/475,847, filed Jun. 7, 1995, now allowed.

One advantage of the present invention is that it enables intervention in an aspect of the immune response which is not antigen specific. Many current therapies for allergy include desensitization to particular antigens, and require that each patient be tested in order to identify antigens associated with sensitivity. As a practical matter, exhaustive analysis of a patient's response to each and every potential allergen is virtually impossible. Furthermore, in most autoimmune conditions, the causative antigen is, generally, unknown or even irrelevant to the disease process. The present invention, which relates to the antigen-nonspecific CD40/CD40CR interaction, circumvents the need to characterize the antigen associated with allergy or autoimmunity. Therefore, the present invention may be used to particular advantage in the treatment of allergic or autoimmune conditions in which the immunogen is not known, or has multiple components, for example, in hay fever, procainamide induced lupus or systemic lupus erythematosus (SLE). It should also be useful in acute treatment of immune activation, for example, in therapy for anaphylaxis.

### Abbreviations

Ig immunoglobulin

mab monoclonal antibody

PM^{ACT} plasma membranes prepared from resting helper T-cells

PM^{REST} plasma membranes prepared from resting helper T-cells

PAGE polyacrylamide gel electrophoresis

rIL4 recombinant interleukin 4

rIL5 recombinant interleukin 5

SN supernatant

Tₕ helper T-cell

Tₕ1 refers to D 1.6, a I-A^{d}-restricted, rabbit immunoglobulin specific clone

### DESCRIPTION OF THE FIGURES

FIG. 1. Effect on monoclonal antibodies and CD40-Ig on the induction of B-cell RNA synthesis by PM^{Act}.

Panel A. Resting B-cells were cultured with Pmtest or PM^{Act} from Tₕ1. 25 μg/ml of anti-CD4, anti-LFA-1 or anti-ICAM-1 or a combination of each of these (each at 25 μg/ml) was added to wells containing PM^{Act} and B-cell RNA synthesis was measured by incorporation of [³H]-uridine. B-cell RNA synthesis was addressed from 42 to 48 hours post-culture. Results presented are the arithmetic means of triplicate cultures +/− s.d., and are representative of 5 such experiments.

Panel B. Resting B-cells are cultured with PM^{Act} from Th1 (•, Ù) or Tₕ2 (o). To the Th¹ PM^{ACT} containing cultures (•, Ù), increasing amounts of CD40-IG (Ù) or control protein CD7E-Ig (•) were added. To the Tₕ2 PM^{Act} containing culture (o), increasing amounts of CD40-Ig were added. B-cell RNA synthesis was assessed from 42 to 48 hours post-culture. Results presented are the arithmetic means of triplicate cultures +/− s.d., and are representative of 3 such experiments.

Panel C. Resting B-cells were cultured with LPS (50 μg/ml) or PM^{Act}. To cultures, CD40-Ig (25 μg/ml; hatched) or CD7E-Ig (25 μg/ml; solid) were added. RNA synthesis was determined as described in Panel A. Results presented are the arithmetic mean of triplicate cultures +/− s.d., and are representative of 3 such experiments.

FIG. 2. CD40-Ig inhibited B-cell differentiation and proliferation.

Panel A. Resting B-cells were cultured with PM^{Act}, rIL4 (10 ng/ml) and rIL5 (5 ng/ml). Either at the initiation of culture, or on days 1, 2 or 3 post-initiation of culture, CD40-Ig or CD7E-Ig (25 μg/ml) were added. On day six of culture, SN from individual wells were harvested and quantitated for IgM (n) and IgG₁ (•) using an anti-isotype specific ELISA, as described in (Noelle et al., J. Immunol., 146:1118-1124(1991)). In the presence of PM^{Act}, IL4 and IL5 (in the absence of added CD40-Ig), the concentrations of IgM and IgG₁ were 4.6 μg/ml and 126 ng/ml of IgM and IgG₁, respectively. In the absence of IL4 and IL5, no IgM or IgG₁ was detected. Results are representative of 3 such experiments.

Panel B. Th1 were rested or activated with anti-CD3 for 16 hours, irradiated and cultured (1×10⁴/well) with resting B-cells (4×10⁴/culture) in the presence of IL4 (10 ng/ml). Between 0 and 25 μg/ml of CD40-Ig (Ù) or CD7E-Ig (•) were added to cultures. From 66-72 hours post-culture, wells were pulsed with 1.0 μCi of [³H]-thymidine and harvested. The dotted line indicates the response of B-cells to resting Th. Results presented ar the arithmetic mean of triplicate cultures +/− s.d., and are representative of 2 such experiments.

FIG. 3. CD40-Ig detected a molecule expressed on activated, but not resting Th. Resting and activated Th were harvested and incubated with fusion proteins for 20 minutes at 4° C., followed by FITC-conjugated goat anti-hIgG (25 μg/ml). Percentage positive cells and MFI were determined by analysis of at least 5000 cells/sample. Results are representative of 6 such experiments. CD40-Ig binding is indicated by a filled-in profile.

FIG. 4. CD40-Ig immunoprecipitated a 39 kD protein from lysate of activated Th1. Th1 was rested

or activated with insolubilized anti-CD3 for 16 hours. [³⁵S]-labelled proteins from resting or activated Th were immunoprecipitated with purified antibodies or fision proteins (1-10μ). The gel profile is representative of 3 such experiments.

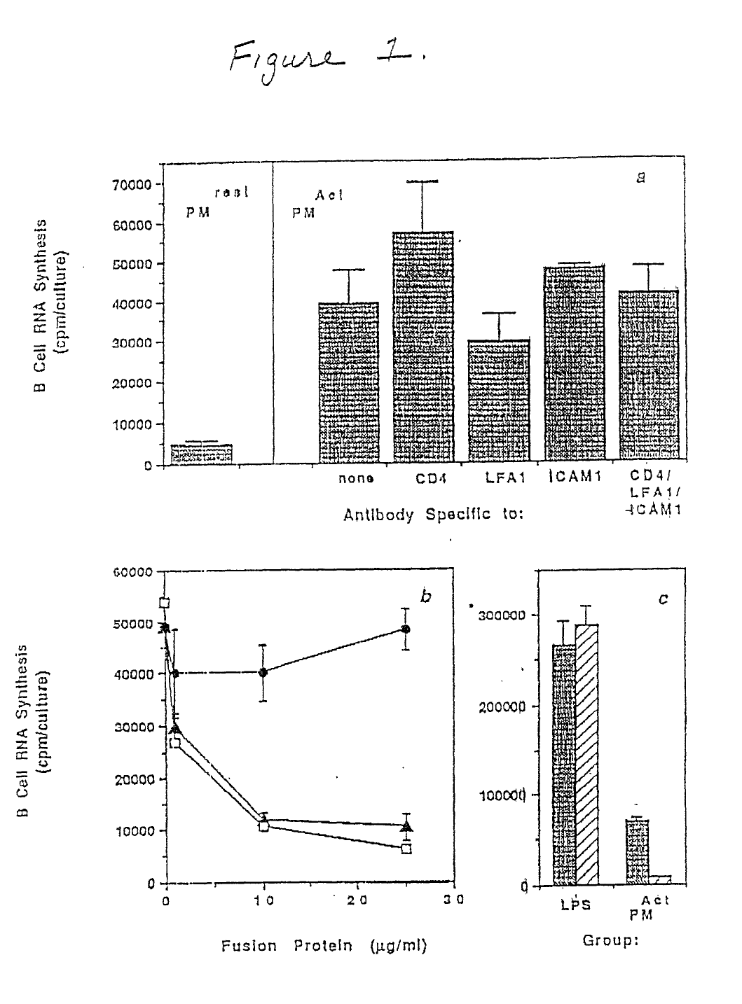

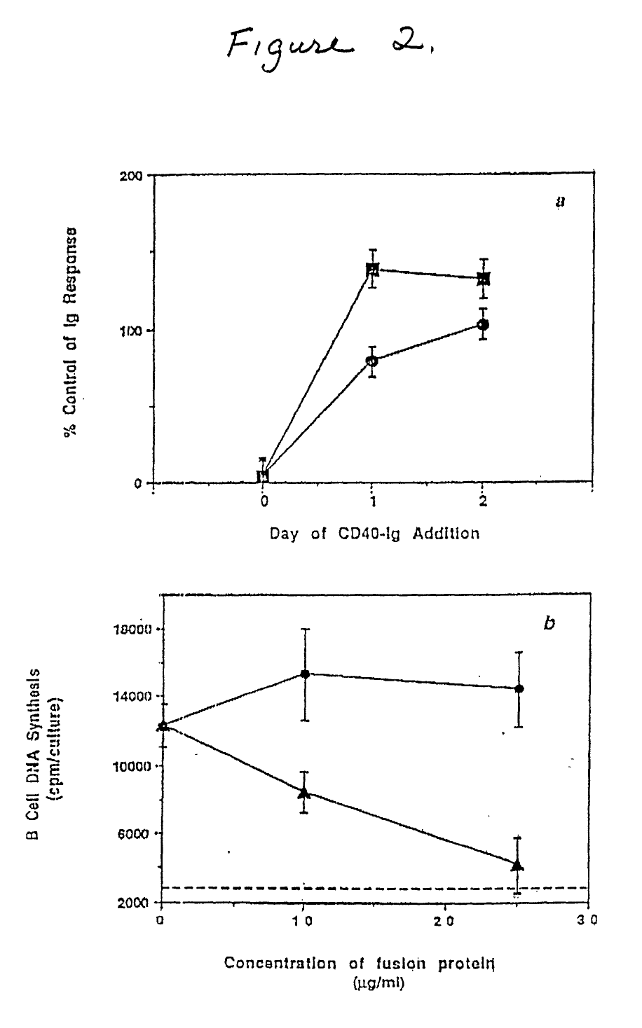

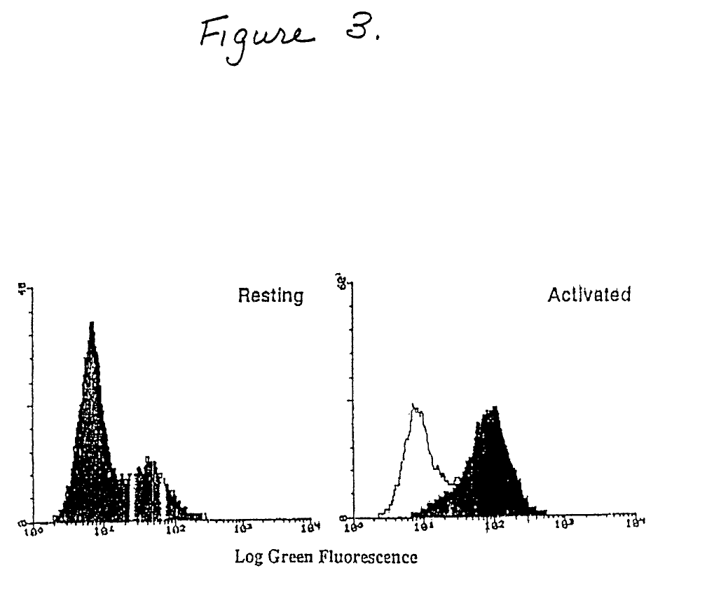

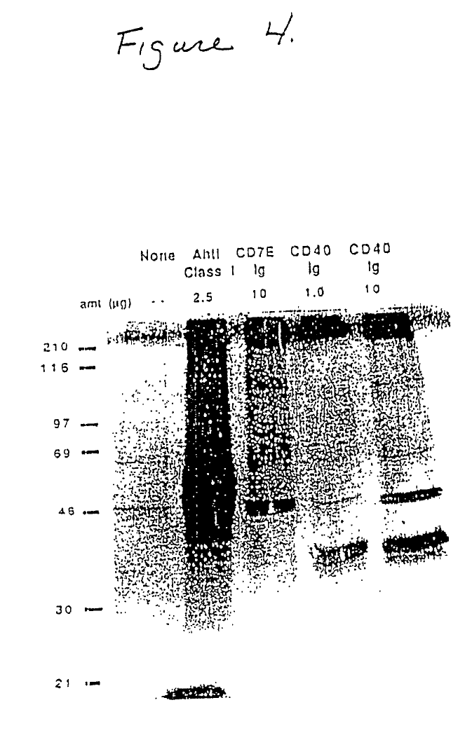

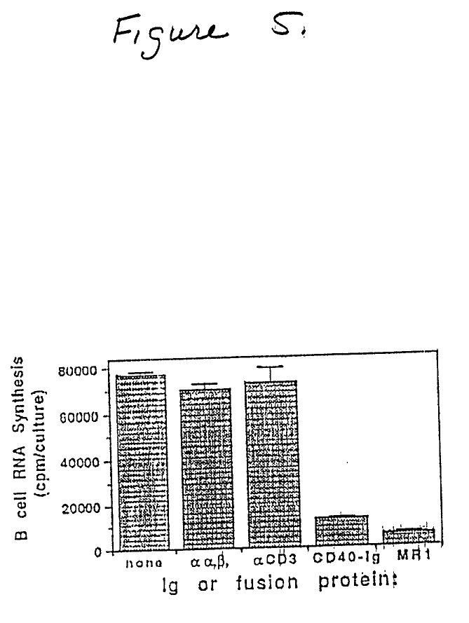

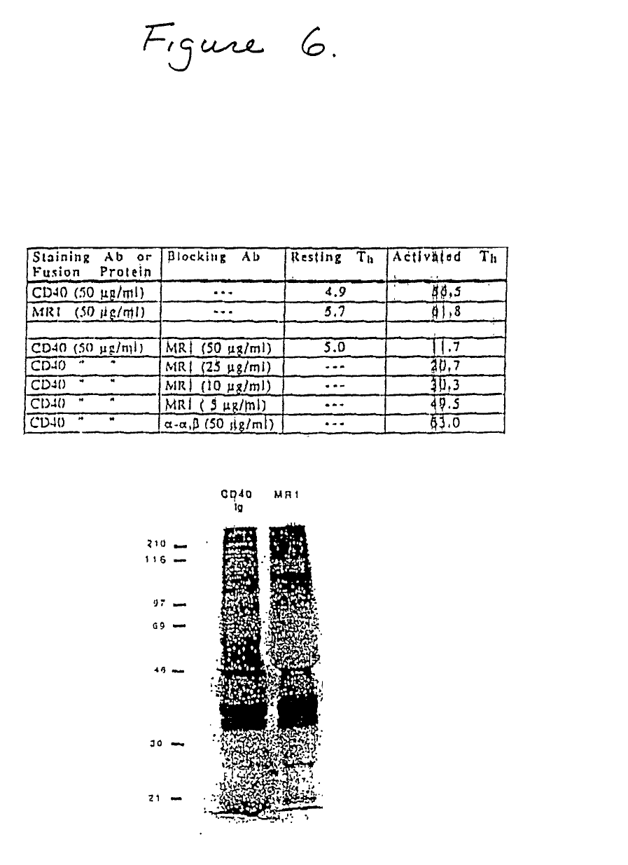

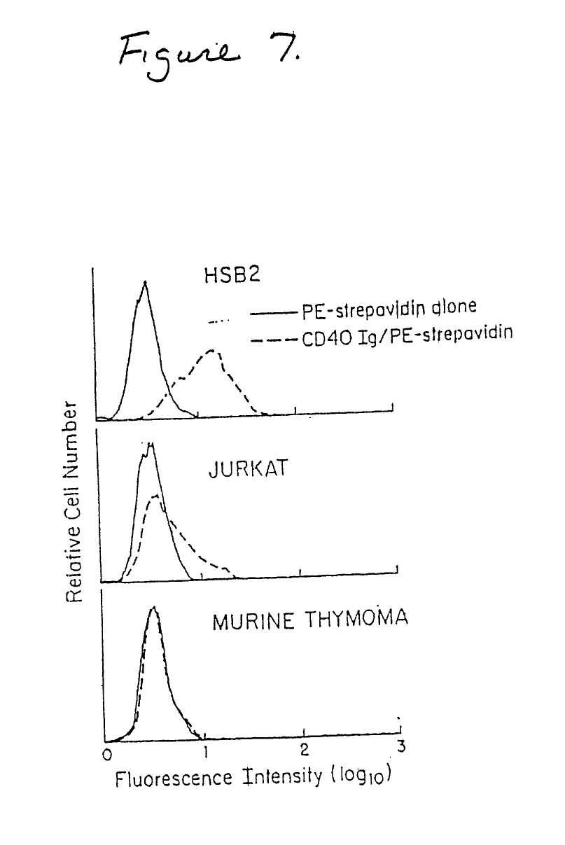

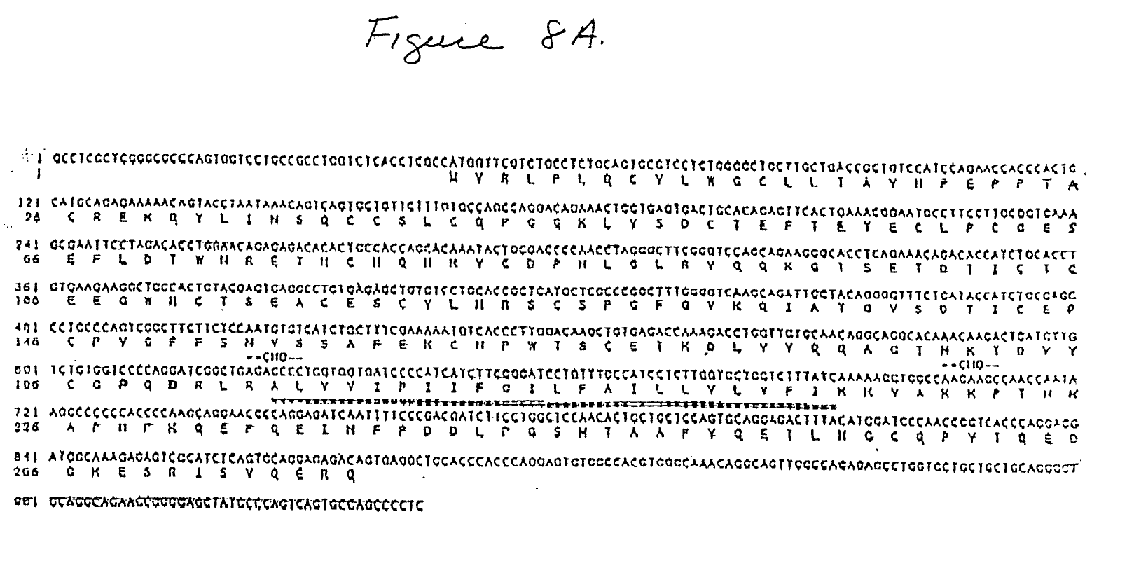

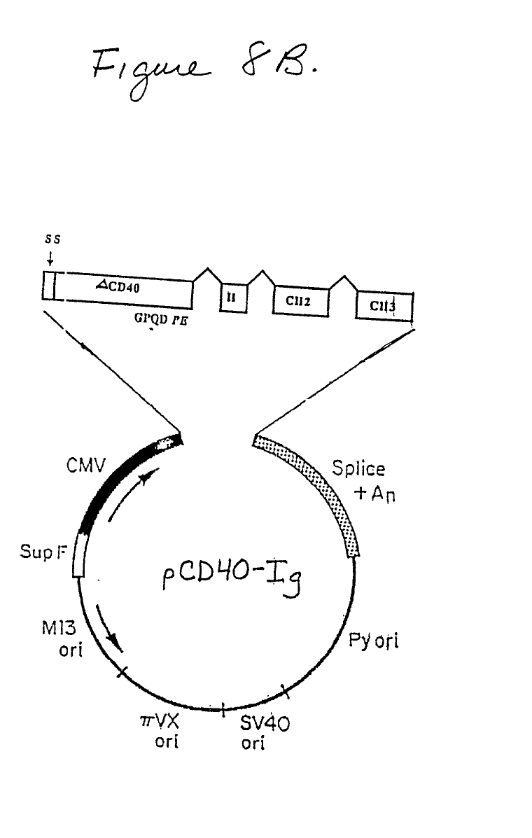

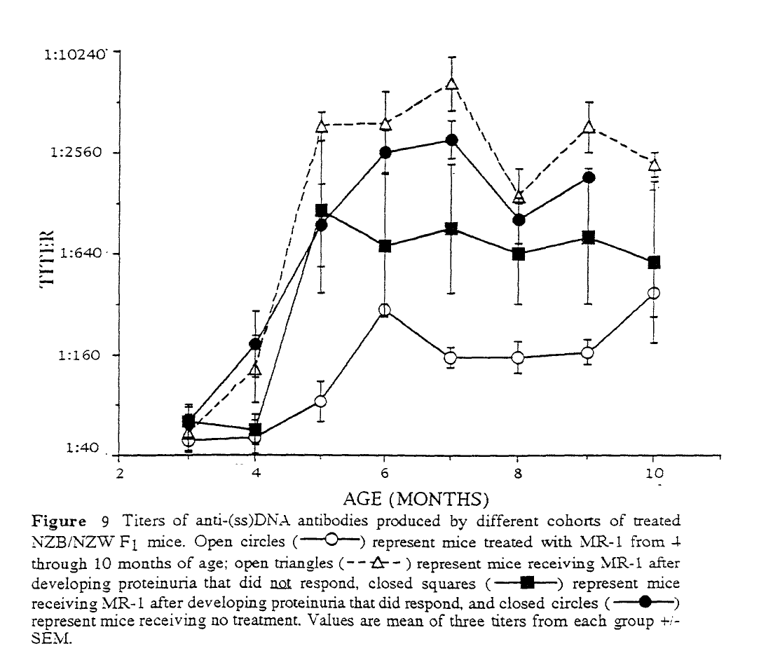

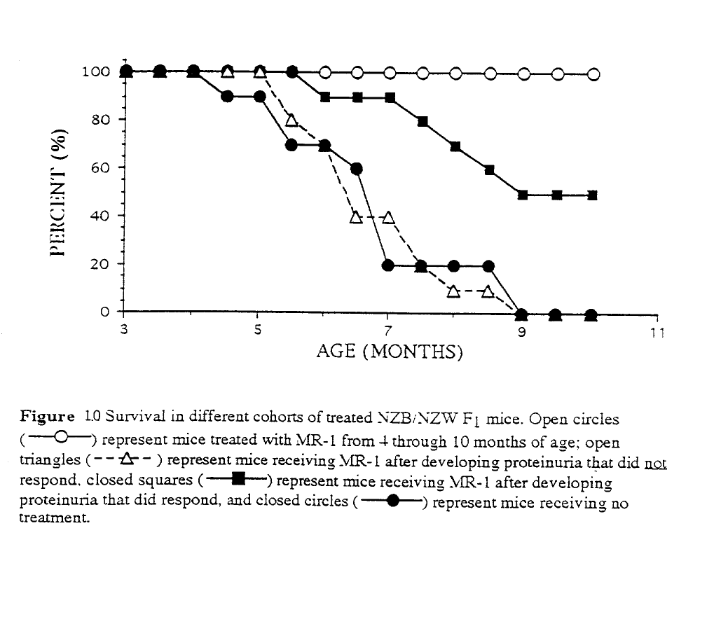

FIG. 5. A monoclonal antibody (mab), specific to the induced 39 Kd Th membrane protein, inhibited induction of B-cell RNA synthesis by PM^{ACT}. Resting B-cells and PM^{ACT} were cultured with 10 μg/ml each of anti-α/β, anti-CD3, CD40-Ig or MR1. RNA synthesis was determined as described in FIG. 1. Results presented are the arithmetic means of triplicate cultures +/− s.d., and are representative of 3 such experiments.

FIG. 6. MR1 and CD40-Ig recognized the same molecule expressed on activated Th.

Panel A: Activated Th were fluorescently stained with MR1 or control Ig. To evaluated if CD40-Ig and MR1 competed for binding to activated Th, graded concentrations of MR1 or control hamster Ig (anti-α/β TCR) were added together with anti-CD40 (20 μg/ml). After incubation for 20 minutes at 4° C., the samples were washed and incubated with FITC-conjugated, mab anti-human IgG₁. Results are representative of 3 such experiments.

Panel B: Proteins from [³⁵S]-methionine-labelled, activated Th were immunoprecipitated with MR1 (10 μg/sample) or CD40-Ig (10 μg/sample) and resolved by PAGE and fluorography. Results presented are representative of 2 such experiments.

FIG. 7. Binding of CD40-Ig to human cell lines. A variety of human T-cell lines were exposed to biotin-labelled CD40-Ig, and binding was evaluated by flow cytometry.

FIG. 8.

Panel A: Nucleotide sequence of CD40 cDNA from Stamenkovic et al., EMBO J., 8:1403-1410 (1989) The transmembrane region is underscored.

Panel B: Schematic diagram of a plasmid that may be used to express CD40-Ig. The amino acid sequences at the site of fusion of A CD40 is shown below the diagramed portion of CD40.

FIG. 9. Titers of anti-(ss)DNA antibodies produced by different cohorts of treated NZB/NZW F₁ mice. Open circles (--- m ---) represent mice treated with MR-1 from 4 through 10 months of age; open triangles (--- Δ ---) represent mice receiving MR-1 after developing proteinuria that did not respond, closed squares (--- n ---) represent mice receiving MR-1 after developing proteinuria that did respond, and closed circles (--- 1 ---) represent mice receiving no treatment. Values are mean of three titers from each group +/− SEM.

FIG. 10. Survival in different cohorts of treated NZB/NZW F1 mice. Open circles (--- m ---) represent mice treated with MR-1 from 4 through 10 months of age; open triangles (--- α ---) represent mice receiving MR-1 after developing proteinuria that did not respond, closed squares (--- n ---) represent mice receiving MR-1 after developing proteinuria that did respond, and closed circles (--- 1 ---) represent mice receiving no treatment.

### DETAILED DESCRIPTION OF THE INVENTION

The present invention provides for a substantially purified CD40CR receptor; for soluble ligands of CD40CR, including anti-gp39 antibodies and fragment thereof, as well as fusion molecules comprising CD40; and for methods of controlling B-cell activation using soluble ligands.

For purposes of clarity of disclosure, and not by way of limitation, the detailed description of the invention is divided into the following subsections:

(i) ligands that bind to CD40CR;

(ii) methods used to characterize CD40CR;

(iii) preparation of purified CD40CR;

(iv) uses of ligands that bind to CD40CR; and

(v) uses of CD40CR.

(iii) preparation of purified CD40CR; (in particular the treatment of ongoing lupus, e.g. systemic lupus erythematosus or drug-induced lupus.

The present invention provides for soluble ligands of CD40CR, including (i) fusion molecules comprising at least a portion of CD40 protein and (ii) antibodies or antibody fragments that specifically bind CD40CR, or gp-39, or CD 154 as such antigen is also known.

The term “soluble,” as used herein, indicates that the ligands of the invention are not permanently associated with a cell plasma membrane. Soluble ligands of the invention may, however, be affixed to a non-cellular solid support, including a lipid, protein, or carbohydrate molecule, a bead, a vesicle, a magnetic particle, a fiber, etc. or may be enclosed within an implant or vesicle.

The ability of such a ligand to bind to CD40CR may be confirmed by demonstrating that the ligand binds to the same protein as CD40-Ig (infra) or MR1 (infra), or another antibody that binds CD40CR, such as disclosed in commonly assigned U.S. Ser. No. 08/475,847.

The ligands of the invention may be comprised in pharmaceutical compositions together with a suitable carrier.

The present invention provides for soluble fusion molecules that are ligands of CD40CR. Such fusion molecules comprise at least a portion of C040 protein attached to a second molecule. The portion of CD40 preferably lacks the CD40 transmembrane domain. A portion of CD40 protein which may be used according to the invention is defined as any portion which is able to bind to CD40CR, for example, such a portion may be shown to bind to the same protein as MR1 or CD40-Ig.

Second molecules which may be used include peptides and proteins, lipids, and carbohydrates, and, in preferred embodiments of the invention, may be an immunoglobulin molecule, or portion thereof (such as an Fv, Fab, F(ab′)₂, or Fab′ fragment) or CD8, or another adhesion molecule, such as B7. The second molecule may be derived from either a non-human or a human source, or may be chimeric. The second molecule may also be an enzyme, toxin, growth factor, lymphokine, antiprolifera-tive agent, alkylating agent, antimetabolite, anti-biotic, vinca alkaloid, platinum coordinated complex, radioisotope, or a fluorescent compound.

The fusion molecules of the invention may be produced by chemical synthesis or, preferably, by recombinant DNA techniques.

For example, a nucleic acid sequence encoding at least a portion of CD40 protein may be combined with a nucleic acid sequence encoding a second molecule in a suitable expression vector, and then expressed in a prokaryotic or, preferably, eukaryotic expression system, such as a yeast, baculovirus, or mammalian expression system, including transgenic animals.

Alternatively, at least a portion of CD40 protein may be expressed using electrophoretic techniques or affinity chromatography using ligand that binds to either CD40 or to the second molecule. Ligands that bind to CD40 include, but are not limited to, anti-CD40 antibodies such as G28-5, as produced by the hybridoma having accession number HB9110 and deposited with the American Type Culture Collection, and CD40CR, described more fully infra. If the second molecule is an immunoglobulin or immunoglobulin fragment, an affinity column comprising anti-immunoglobulin antibody may be used; if the second molecule comprises an F_{c} fragment, a protein A column may be used.

According to a preferred embodiment of the invention, a portion of CD40 may be produced using nucleic acid sequence that encodes a CD40 protein that is truncated upstream from the transmembrane domain. Such a nucleic acid sequence may be prepared by digesting a plasmid containing a cDNA encoding CD40 antigen, such as that described in Stamenkovic et al., EMBO J, 8:1403-1410 (1989), with PstI (P) and Sau 3A (S3) restriction enzymes. The resulting P/S3 fragment may be subcloned into the same plasmid digested with P and Bam HI (B), to produce a truncated CD40 gene (see FIG. 8).

In particular, nonlimiting, embodiments of the invention, an expression vector used to produce ligands containing at least a portion of CD40 as well as immunoglobulin sequence may preferably comprise a virally-derived origin of replication, a bacterial origin of replication, a bacterial selectable marker, and eukaryotic promoter and enhancer sequences separated from DNA sequences encoding an immunoglobulin constant region by restriction endonuclease sites which allow subcloning of DNA sequences encoding at least a portion of CD40, followed by a polyadenylation signal sequence (see FIG. 8.b.).

In a specific embodiment of the invention, the truncated CD40 gene may be subcloned into an immuno-globulin fusion plasmid, such as that described in Aruffo et al., Cell, 61:1303-1313 (1990), using an Mlu I and B digest, to form plasmid pCD40-Ig, which encodes the fusion molecule CD40-Ig (see FIG. 8). CD40-Ig fusion protein may then be produced by transfecting the PCD40-Ig plasmid into COS cells to form a transient expression system. CD40-Ig produced may be collected from the COS cell supernatant and purified by protein A column chromatography as described in Aruffo et al., Cell, 161:1303-1313 (1990).

The soluble ligands of the invention will preferably comprise antibody molecules, monoclonal antibody molecules, or fragments of these antibody molecules which contain an antigen combining site that binds to CD40CR (gp39) preferably human gp39. Such ligands may further comprise a second molecule which may be a protein, lipid, carbohydrate, enzyme, toxin, growth factor, lymphokine, antiproliferative agent, alkylating agent, antimetabolite, antibiotic, vinca alkaloid, platinum coordinated complex, radioisotope, or a fluorescent compound and may be linked to the antibody molecule or fragment.

Where the ligand is a monoclonal antibody, or a fragment thereof, the monoclonal antibody can be prepared against CD4CR (gp39) using any technique which provides for the production of antibody molecules by continuous cell lines in culture. For example, the hybridoma technique originally developed by Kohler and Milstein (1975, Nature, 256:495-497) as well as other techniques which have more recently become available, such as the human B-cell hybridoma technique (Kozbar et al., 1983, Immunology Today, 4:72)) and EBV-hybridoma technique to produce human monoclonal antibodies (Cole et al., 1985, Monoclonal Antibodies and Cancer Therapy, Alan R. Liss, Inc., pp 77-96) and the like are within the scope of the present invention. Humanized and chimeric anti-gp39 antibodies are generally preferred because they are less prone to eliciting immunogenic responses (HAMA responses) upon administration.

Antibody fragments which contain the idiotype of the molecule can be generated by known techniques. For example, such fragments include but are not limited to: the F(ab′)₂ fragment which can be generated by treating the antibody molecule with pepsin; the Fab′ fragments which can be generated by reducing the disulfide bridges of the F(ab′)₂ fragment; the F(ab′)₂ fragment which can be generated by treating the antibody molecule with papain; and the 2Fab or Fab fragments which can be generated by treating the antibody molecule with papain and a reducing agent to reduce the disulfide bridges.

As noted above, the present invention also provides for chimeric or human antibodies produced by techniques known in the art, such as those set forth in Morrison et al., Proc. Natl. Acad. Sci. U.S.A., 81:6851-6855 (1984) or European Patent Application No. 85305604.2, publication No. 0173494 by Morrison et al., published Mar. 5, 1986.

Immunogen for the production of antibodies may be any source that contains CD40CR. For example, activated Tₕ, e.g. activated human Tₕ cells may be used as an immunogen. Alternatively, substantially purified CD40CR, prepared as set forth in section 5.3, infra, may be used. If activated Th are used as immunogen, antiserum may be tested for reactivity against activated but not resting Tₕ cells.

Also, the immunogen may comprise recombinant gp39 or a fragment thereof. In this regard, the DNA's encoding both murine and human gp39 have been cloned and expressed by recombinant methods. These proteins provide a potential immunogen for producing anti-gp39 antibodies.

In a preferred embodiment of the invention, the soluble ligand is an anti-human gp39 monoclonal antibody, more preferably a humanized or chimeric anti-human gp39 antibody. The following method was used to produce the MR1 monoclonal antibody, which specifically binds murine gp39 and may be used to generate other antibodies directed toward CD40CR.

Hamsters were immunized intraperitoneally with 5-10⁶ activated Tₕ1 cells (D1.6) at weekly intervals for six weeks. When the serum titer against murine Tₕ1 was greater than about 1:10,000, cell fusions were performed with polyethylene glycol using immune hamster spleno-cytes and NSI. SN from wells containing growing hybridomas were screened by flow cytometry on resting and activated Tₕ1. One particular hybridoma, which produced a mab that selectively recognized activated Th, was further tested and subcloned to derive MR1. MR1 was produced in ascites and purified by ion exchange HPLC.

Alternatively, and more preferably, antibodies against human gp39 can be prepared according to U.S. Ser. No. 08/475,847, incorporated by reference in its entirety herein.

The present invention also provides for ligands comprising monoclonal antibodies, and fragments thereof that are capable of competitively inhibiting the binding of MR1 to its target antigen or CD40-Ig to its receptor.

CD40CR may be characterized by (i) its ability to bind CD40, fusion molecules comprising at least a portion of CD40, and antibodies such as MR1; (ii) its functional characteristic of being able to stimulate B-cell cycle entry, proliferation, and differentiation, and (iii) its cellular distribution.

CD40CR may be characterized by its ability to bind to ligands such as CD40, fusion molecules comprising CD40, and antibodies directed toward CD40CR.

As discussed in greater detail infra, several techniques were used to characterize CD40CR. For example, CD40-Ig and MR1 were shown to recognize the same 39 kD molecule. Both CD40-Ig and MR1 were found to immunoprecipitate a 39 kD protein from radiolabeled Th lysates (FIG. 5b). Further, immunoprecipitation of the 39 kD protein with CD40-Ig removed the antigen recognized by MR1 from Th lysates.

CD40CR may also be characterized by its ability to stimulate B-cell cycle entry, proliferation, and differentiation.

For example, plasma membrane (PM) from activated (PM^{ACT}) but not resting (PM^{REST}) Th cells was found to induce B-cell RNA synthesis (FIG. 1a); this induction, indicative of B-cell activation, was not affected by antibodies such as anti-LFA-1, anti-CD4, anti-ICAM-1. CD40-Ig or MR1, however, were found to be able to inhibit PM^{ACT}-induced B-cell activation, as shown in FIG. 1b and FIG. 6.

The induction of B-cell activation may be measured by techniques such as [³H]-uridine incorporation into RNA (as B-cells differentiate, RNA synthesis increases), or by [³H]-thymidine incorporation, which measures DNA synthesis associated with cell proliferation. For optimal measurement of the effect of CD40CR on B-cell proliferation, interleukin-4 (IL-4) may be added to the culture medium at a concentration of about 10 ng/ml.

Alternatively, B-cell activation may be measured as a function of immunoglobulin secretion. For example, CD40CR, in substantially purified form, or as present in PM, or otherwise, may be added to resting B-cells together with IL-4 (10 ng/ml) and IL-5 (5 ng/ml). After three days of culture, an additional volume of culture medium may be added. On day 6 of culture, supernatant (SN) from individual cultures may be harvested and quantitated for IgM and Ig₁ as described in Noelle et al.,J. Immunol., 146:1118-1124 (1991).

CD40CR may also be characterized by its cellular distribution. For example, CD40-Ig was observed to bind to activated, but not resting Tₕ1, as assessed by flow cytometry (FIG. 3). Furthermore, CD40-Ig was observed to bind to Jurkat cells, HSB2 cells, and activated T-cells from human peripheral blood, but did not appear to bind significantly to CEM cells, HPBALL cells, or murine thyoma cells.

For example, and not by way of limitation, the presence of CD40CR on a particular cell type (“test cells”) may be evaluated by flow cytometry as follows. Test cells may be tested in parallel with resting (negative control) and activated (positive control) Th cells. All cells may be incubated at a concentration of about 1×10⁵ cells/50 μl with ligand (e.g. CD40-Ig or MR1) for 20 minutes at 4° C., followed by FITC-conjugated anti-ligand antibody. Propidium iodide may be added to all samples to a final concentration of 2 μg/ml. Flow cytometric analysis may then be performed, for example on a BD FACSCAN. After positive gating of cells by forward versus side scatter, and by red negativity (for propidium iodide exclusion), and the log green fluorescense of viable cells may be ascertained.

The present invention provides for substantially purified CD40CR. Such CD40CR may be prepared from cells bearing CD40CR, such as activated helper T-cells, Jurkat, and HSB2 cells, by the following method.

Plasma membranes may be prepared from appropriate cells, such as activated Tₕ1 cells, by discontinuous sucrose gradient sedimentation, as described in Noelle et al., J. Immunol., 146:1118-1124 (1991). CD40CR may then be isolated by dissociating the crude membrane extract with mild detergent, and then performing size exclusion chromatography followed by either affinity chromatography using appropriate ligands (e.g. MR1 or CD40-Ig) bound to a solid support, immunoprecipitation (e.g. by CD40-Ig or MR1), and/or gel electrophoresis.

The resulting protein may be expected to have a molecular weight of about 39 kD.

The present invention provides for a soluble CD40CR (i.e. cell-free) which may be comprised in pharmaceutical compositions together with a suitable carrier. It further provides for CD40 CR which is linked to a second molecule which may be a peptide, protein, lipid, carbohydrate, enzyme, toxin, growth factor, lymphokine, antiproliferative agent, alkylating agent, antimetabolite, antibiotic, vinca alkaloid, platinum coordinated complex, radioisotope, or a fluorescent compound.

The present invention further provides for substantially purified CD40CR which has been prepared by chemical synthesis or recombinant DNA techniques. For example, the gene for CD40CR may be isolated by inserting cDNA prepared from activated helper T-cells into the λgt10 expression system, and then screening with MR1 or CD40-Ig binding to identify CD40CR-expressing clones. Alternatively, cDNA prepared from activated helper T-cells may be transfected into COS cells, the supernatants of which may be screened with MR1 or CD40-Ig to identify CD40CR producers. The gene for CD40CR may be then used to express CD40CR using expression systems known in the art.

The present invention provides for methods of controlling B-cell activation that utilize ligands that bind to CD40CR. h particular, it provides for a method of inhibiting B-cell activation comprising exposing a mixture of B-cells and Th cells to an effective concentration of ligand that binds to CD40CR. Ligands that may be used are described supra in section 5.1. The method of the invention may be practiced in vitro or in vivo. An effective concentration refers to a concentration of a ligand that inhibits B-cell activation, measured by any technique known in the art (including those set forth in section 5.2, supra) by at least about 30 percent, and preferably by about 75 percent. According to a preferred, specific, non-limiting embodiment of the invention, CD40-Ig may be used as ligand, in which case an effective concentration may be at least about 10 μg/ml. In another specific, nonlimiting embodiment of the invention, the monoclonal antibody MR1 may be used, in which case an effective concentration may be at least about 10 μg/ml. If the method is practiced in vivo, an effective concentration of ligand may refer to plasma concentration of ligand or to a local concentration. For example, it may be desirable to inhibit B-cell activation in a localized area in order to limit the effects on the immune system as whole.

In particular embodiments, the invention provides for a method of treating a subject suffering from a disorder associated with B-cell activation, comprising administering to the subject a therapeutic amount of ligand that binds to CD40CR. A subject may be a non-human or, preferably, a human animal.

Disorders associated with B-cell activation include, but are not limited to, allergy (including anaphylaxis); autoimmune conditions including drug induced lupus, systemic lupus erythematosus, adult rheumatoid arthritis, juvenile rheumatoid arthritis, scleroderma, Sjogren's Syndrome, etc.; and viral diseases that involve B-cells, including Epstein-Barr infection, and retroviral infection including infection with a human immunodeficiency virus.

Because it has been suggested that B-cell activation is associated with the induction of human immunodeficiency virus replication from latency, it may be desirable to administer the ligands of the invention to HIV positive individuals who have not yet developed AIDS or ARC.

As discussed above, and in the examples which follow, a particularly preferred application of anti-gp39 antibodies, or fragments thereof, comprises their use for the treatment of drug-induced lupus or systemic lupus erythematosus. It has been discovered, as substantiated by the underlying data and experiments in the examples which follow, that anti-gp39 antibody therapy reduces autoantibody production, and renal disease, and results in prolonged survival in NZB/NZW, an accepted animal model for human SLE.

Moreover, it has further been shown in mice allowed to develop 2-3+ proteinuria (active lupus disease condition), that the administration of anti-gp39 antibody actually reversed disease (as evidenced by prolonged survival and absence of proteinuria after antibody administration). Thus, treatment with anti-gp39 antibodies was shown, after development of renal disease in an accepted animal model of human lupus, to reverse the lupus disease process. This substantiates the potential of anti-gp39 antibodies for human therapeutic applications in the treatment of lupus and other autoimmune diseases. In particular, it substantiates that such antibodies may be used to treat persons having an active ongoing disease, even at advanced stages of the disease. Such treatment will effectively treat and potentially even reverse the disease process, e.g, the renal damage which often results in patients with lupus.

Ligands may be administered, in a suitable pharmaceutical carrier, by any method known in the art, including intravenous, intraperitoneal, subcutaneous, intrathecal, intraarticular or intramuscular injection, and oral, intranasal, intraocular and rectal administration, and may be comprised in microspheres, liposomes, and/or sustained release implants.

A therapeutic amount of ligand is defined as an amount which significantly diminishes the deleterious clinical effects of B-cell activation or T cell activation, and may vary among ligands used and conditions treated. If CD40-Ig is used, therapeutic concentration may be about 10 μg/ml either systemically (plasma concentration) or locally. If MR1 or another anti-gp39 antibody, e.g., a humanized or chimeric anti-human gp39 antibody or fragment thereof, is used, a therapeutic concentration may be about 10 μg/ml either systemically (plasma concentration) or locally.

In a further embodiment of the invention, the above methods may utilize a ligand comprising a toxin or antimetabolite such that Th cells are killed or damaged and B-cell activation is increased as a result of Th cell destruction.

The ligands of the invention may also be used to label activated T cells, a technique which may be useful in the diagnosis of T cell disorders. To this end, ligand comprising an enzyme, radioisotope, fluorescent compound or other detectable label may be exposed to T cells in vitro or in vivo and the amount of binding may be quantitated.

The ligands of the invention may also be used to deliver substances, e.g. growth factors, to activated T-cells.

The present invention provides for methods of controlling B-cell activation that utilize CD40CR or a molecule comprising CD40CR, prepared as described supra. In particular, it provides for a method of promoting B-cell activation comprising exposing B-cells to an effective concentration of CD40CR. The method may be practiced in vivo or in vitro. An effective concentration refers to a concentration of receptor that induces B-cell activation, measured by any technique known in the art by at least about 30 percent. In specific, nonlimiting embodiments of the invention, the concentration of CD40CR may be about 10 μg/ml locally or systemically.

In particular embodiments, the invention provides for a method of treating a subject suffering from an immunodeficiency disorder associated with diminished humoral immunity, comprising administering to the subject a therapeutic amount of CD40CR. A subject may be a non-human or, preferably, a human animal.

Immunodeficiency disorders associated with diminished humoral immunity include acquired immunodeficiency caused, for example, by chemotherapy or radiation therapy, as well as genetic disorders involving humoral immunity.

CD40CR may be administered, in a suitable pharmaceutical carrier, by any method known in the art, including intravenous, intraperitoneal, subcutaneous, intrathecal, intraarticular, or intramuscular injection, and oral, intranasal, intraocular, and rectal administration and may be comprised in microspheres, liposomes, and/or sustained release implants.

A therapeutic amount of CD40CR for CD40 is defined as that amount which increases immunoglobulin production by at least about 30 percent.

In a further embodiment, a CD40CR may be conjugated to a toxin, and then administered to a subject under circumstances in which it would be preferable to destroy B-cells that express CD40. Examples of such circumstances include patients receiving organ transplants or suffering from multiple myeloma or another B-cell malignancy, or from autoimmune disease.

CD40CR may also be used to label B-cells expressing CD40, a technique which may be useful in the diagnosis of B-cell disorders. To this end, receptor linked to an enzyme, radioisotope, fluorescent compound or other detectable label may be exposed to B-cells in vivo or in vitro and the amount of binding may be quantitated.

CD40CR may also be used to deliver molecules that are linked to it to B-cells.

### EXAMPLE 1

### A Novel Receptor, CD40CR, on Activated Helper T-cells Binds CD40 and Transduces the Signal for Cognate Activation of B-cells

Materials and Methods

Animals.

Female DBA/2J mice (Jackson Laboratories, Bar Harbor, Me.) were used for the preparation of filler cells to support the growth of Th clones and in the preparation of resting B-cells.

D1.6, a I-Ad-restricted, rabbit Ig-specific Tₕ1 clone (Kurt-Jones et al., J. Exp. Med., 166:1774-1787 (1987)) was obtained from Dr. David Parker, University of Massachusetts at Worcester. D1.6 will be referred to herein as Tₕ1.

Tₕ1 were cultured (8×10⁶/well) in cluster wells (6 well, Coming, N.Y.) coated with 40 μg/4 ml of PBS/well with anti-CD3 for 16 hours, as described in Noelle et al. (J. Immunol., 146:1118-1124 (1991)).

Plasma membranes were prepared by discontinuous sucrose gradient sedimentation, as described in Noelle et al. (Id.).

Resting splenic B-cells were prepared by sedimentation on discontinuous Percoll gradients, as described in Defranco et al. (J. Exp. Med., 155:1523 (1982)). Cells isolated from the 70-75% (density of 1.087-1.097) Percoll interface were typically >95% mIg⁺, had a uniform, low degree of near forward light scatter and were unresponsive to Con A.

The following mabs were purified by ion exchange HPLC from ascites fluid of mice which had been irradiated and bone marrow reconstituted: anti-CD3:145-2C11 (Leo et al., Proc. Natl. Acad. Sci. USA, 84:1374-1378 (1987); anti-α,β:H57-597; anti-CD4:GK1.5 (Wilde et al., J. Immunol., 131:2178-2183 (1983); anti-ICAM:YN1/1.7.4 (Prieto et al., Eur. J. Immunol., 19:1551-1557 (1989)); anti-LFA-1: FD441.8 (Sarmiento et al., Immunol. Rev., 68:135 (1982)); and anti-rat/hamster K chain:RG-7 (Springer, Hybrid., 1:257-273 (1982)).

The CD40 fusion protein was prepared by digesting a plasmid containing a cDNA encoding the CD40 antigen (Stamenkovic and Seed, EMBO J., 8:1403-1410 (1989)) with the restriction enzyme Pst I (P) and Sau 3A (S3). This P/S3 fragment was subcloned into the same plasmid digested with P and Bam H1 (B). This allowed the preparation of the CD40Δ which encoded a CD40 protein truncated upstream from the transmembrane domain. The DNA fragment encoding a CD40Δ was then subcloned into the immunoglobulin fusion plasmid (Aruffo et al., Cell, 61:1303-1313 (1990)) using a MluI and B digest. The CD40-Ig fusion protein was produced by transient transfection in COS cells and purified on a protein A column as described in Aruffo et al (Cell, 61:1303-1313 (1990)).

Interleukin 4 (IL4): Recombinant mouse IL4 was generously provided by Drs. C. Maliszewski and K. Grabstein, Immunex Corporation, Seattle, Wash.

Interleukin 5 (IL5): Recombinant mouse IL5 was purchased from R&D Research, Sarrento, Calif.

3×10⁴ resting B-cells were cultured in 50 μl of cRPMI in A/2 microtiter wells (Costar, Cambridge, Mass.). To these wells, 0.5 μg of Tₕ1 or Tₕ2 membrane protein was added. From 42-48 hrs, wells were pulsed with 2.5 μCi of ³H-uridine (New England Nuclear, Boston, Mass.), harvested, and the radioactivity determined by liquid scintillation spectroscopy. The results were expressed as cpm/culture +/− s.d.

Resting B-cells were cultured as described above. To culture wells, 0.5 μg of Tₕ1 membrane protein, IL4 (10 ng/ml) and IL5 (5 ng/ml) were added. On day three of culture, an additional 50 μl of cRPMI was added. On day six of culture, SN from individual wells were harvested and quantitated for IgM and IgG₁, as described in Noelle et al. (J. Immunol., 146:1118-1124 (1991)).

4×10⁴ resting B-cells were cultured in 50 μl of cRPMI in A/2 microtiter walls (Costar, Cambridge, Mass.). To these wells, 1×10⁴ resting or activated, irradiated (500 rads) Tₕ1 and IL4 (10 ng/ml) were added. On day three of culture, wells were pulsed with 1 μCi of ³H thymidine, as described in (Noelle et al., (1991) i J. Immunol. 146:1118-1124).

Hamsters were immunized intraperitoneally with 5-10×106 activated Tₕ1 (D1.6) at weekly intervals for six weeks. When the serum titer against murine Tₕ1 was greater than 1:10,000, cell fusions were performed with polyethylene glycol using immune hamster splenocytes and NS1. SN from wells containing growing hybridomas were screened by flow cytometry on resting and activated Tₕ1. One particular hybridoma, which produced a mab that selectively recognized activated Tₕ, was further tested and subcloned to derive MR1. MR1 was produced in ascites and purified by ion exchange HPLC.

Resting and activated Tₕ (16 hours with anti-CD3) were harvested and incubated at 1×10⁵ cells/50 μl with fusion protein for 20 minutes at 4° C., followed by FITC-conjugated goat anti-human (h) IgG (25 μg/ml; Southern Biotechnology, Birmingham, Ala.). To all samples, propidium iodide was added at final concentration of 2 μg/ml. Flow cytofluorometric analysis was performed on a BD FACSCAN. After positive gating of cells by forward versus side scatter, and by red negativity (for propidium iodide exclusion), the log green fluorescence of viable cells was ascertained. At least 5,000 viable cells were analyzed for the determination of percent positive cells and MFI. Staining with MR1 employed FITC-conjugated RG7, a mouse anti-rat/hamster K chain mab.

Tₕ1 were rested or activated with insolubilized anti-CD3 for 16 hrs. Proteins from resting and activated Th (20×10⁶/ml) were labelled with 1 mCi of [³⁵S] methionine/cysteine for one hour, at which time they were washed twice in RPMI/10%FCS and the cell pellet was lysed in extraction buffer, as described (Noelle et al., (1986) J. Immunol. 137:1718-1726). Purified antibodies or fusion proteins (1-10 μg) were added to 500 μl of lysate (5×10⁶ cell equivalents) at 4° C. for 16 hours. At that time, the lysates were transferred to tubes containing 50 μl of packed Protein A-sepharose. The pelleted Protein A-Sepharose was resuspended and tubes were incubated at 4° C. for 1 hr with agitation. The samples were then washed 3× with high stringency wash buffer. The pelleted protein A-Sepharose was resuspended in 30 μl of SDS sample buffer and run on a 10% polyacrylamide gel. After running the gel, the gel was fixed and fluorography performed.

In order to define the cell surface molecules that mediated the induction of B-cell cycle entry by PM^{ACT}, mabs to Tₕ membrane proteins were added to cultures of PM^{ACT} and B-cells. PM^{ACT} induced B-cell RNA synthesis eight-fold over that observed with PM^{REST}) (FIG. 1a). The addition of anti-LFA-1, anti-CD4, anti-ICAM-1, alone, or in combination, did not inhibit the induction of B-cell RNA synthesis by MP^{Act}.

In the human system, it had been shown that anti-CD40 mab induced B-cell proliferation (Clark and Lane, (1991) Ann. Rev. Immunol. 9:97-127) thereby implicating CD40 as an important triggering molecule for B-cells. To determine if CD40 was involved in the induction of B-cell RNA synthesis by PM^{ACT}, a soluble fusion protein of the extracellular domains of human CD40 and the F_{c} domain of human IgG₁ (CD40-Ig) was added to cultures of PM^{ACT} and B-cells. PM^{ACT} derived from Tₕ1 and Tₕ2 were prepared and used to stimulate B-cell RNA synthesis. The addition of CD40-Ig to culture caused a dose-dependent inhibition of B-cell RNA synthesis that was induced by PM^{ACT} from Tₕ1 and Tₕ2 was about 5 μg/ml CD40-Ig. A CD7E-Ig fusion protein (Damle and Aruffo, (1991) Proc. Natl. Acad. Sci. USA 88:6403-6407) was without effect even when used at 25 μg/ml.

To investigate whether CD40-Ig inhibited the activation of B-cells by T-independent activators, B-cells were cultured in the presence of LPS and CD40-Ig. On day 2, RNA synthesis was assessed (FIG. 1c). CD40-Ig was ineffective at inhibiting B-cell activation by LPS, yet inhibited the response of B-cells to PM^{ACT}.

In the presence of PM^{ACT}, IL4 and IL5, B-cells polyclonally differentiated to produce Ig (Hodgkin et al., (1990) J. Immunol. 145:2025-2034; Noelle et al, (1991) J. Immunol. 146:1118-1124). To evaluate the requirements for CD40 signalling in this process, CD40-Ig was added at the initiation of culture, or on subsequent days of culture. The addition of CD40-Ig (FIG. 2a) at the initiation of culture inhibited greater than 95% of polyclonal IgM and IgG₁ production compared to control levels in its absence. In contrast, the addition of CD40-Ig on day 1 and 2 of culture showed little, if any, inhibitory effect on IgM and IgG₁ production. These data indicated that after 24 hours signalling via CD40 is no longer essential for the differentiation of B-cells to Ig secretion.

Data thus far indicated that CD40 was implicated in the activation of B-cells by PM^{ACT}. Studies were performed in order to ensure that CD40 was also involved in the activation of B-cells by intact, viable, activated Tₕ. Tₕ1 were activated for 16 hours with insolubilized anti-CD3, harvested and irradiated. The irradiated Tₕ1 were cultured with B-cells in the presence of IL4 and B-cell proliferation was determined on day 3 of culture. An exogenous source of IL4 was required to achieve B-cell proliferation with Tₕ1, because Tₕ1 do not produce IL4 (Noelle et al, (1989) J. Immunol. 143:1807-1814). CD40-Ig inhibited the induction of B-cell proliferation by irradiated Tₕ in a dose-dependent manner, similar to that observed with PM^{ACT} (FIG. 2b). The negative control, CD7E-Ig, exerted no appreciable effect.

To investigate whether activated Tₕ1 express a binding protein for CD40, resting and activated (16 hours) Tₕ1 were stained with CD40-Ig or CD7E-Ig, followed by FITC-anti-HigG. Binding of CD40-Ig was assessed by flow cytometry (FIG. 3). Tₕ1 that were activated for 16 hours with anti-CD3, but not resting Tₕ1, stained 56% positive with CD40-Ig, but not with the control CD7E-Ig. To identify the CD40-Ig binding protein, Tₕ1 proteins were biosynthetically labelled with [³⁵S]-methionine/cysteine and proteins immunoprecipitated with CD40-Ig or CD7E-Ig. The immunoprecipitated proteins were resolved by SDS-PAGE and fluorography (FIG. 4). A prominent band with an apparent molecular weight of 39 kD and a low molecular weight band, β2 microglobulin. In the absence of mab, no prominent bands were visible. A 39 kd band was also immunoprecipitated from activated Th that were vectorially labelled with ¹²⁵I, confirming that the 39 kD protein was a membrane protein.

Mabs specific to antigens selectively expressed on activated versus resting Th were developed to identify Th molecule(s) responsible for the Tₕ effector phase activity. One such mab, MR1, recognized an antigen that was selectively expressed on activated Tₕ1. To investigate whether MR1 and CD40-Ig recognized the same molecule, flow cytometry and blocking studies were performed. CD40-Ig and MR1 stained approximately 56% and 61%, respectively, of activated, but not resting Th (FIG. 5a). MR1, but not another hamster anti-T cell mab, anti-α/β TCR, blocked the staining of activated Tₕ1 with CD40-Ig, in a dose-dependent manner. These data suggested that CD40-Ig and MR1 recognized overlapping or identical epitopes on the 39 kD Th protein. To further demonstrate that CD40-Ig and MR1 recognized the same molecule, the antigen that bound MR1 was identified by immunoprecipitation of proteins from radiolabelled Th lysates. Both CD40-Ig and MR1 immunoprecipitated a 39 kD protein (FIGS. 1. 5b). Finally, immunoprecipitation of the 39 kD protein with CD40-Ig removed the antigen recognized by MR1 from radiolabelled lysates of activated Tₕ supporting the tenet that the MR1 antigen and the CD40 binding protein were identical.

Functional studies were performed with MR1 to address whether this mab neutralized the activity expressed by PM^{ACT}. PM^{ACT} and B-cells were cultured alone, or in the presence of hamster mabs or CD40-Ig. Two hamster mabs, anti-α/β TCR and α-CD3 did not inhibit the activation of resting B-cells by PM^{ACT}. In contrast, MR1 or CD40-Ig inhibited B-cell activation (FIG. 6).

The data show that blocking of prominent Th surface molecules (LFA-1, CD4, ICAM-1, CD3, α,β TCR) with mabs did not impede the capacity of activated Tₕ to induce B-cell cycle entry. In contrast, CD40-Ig or a maB specific to the CD40 binding protein, blocked Tₕ-dependent B-cell activation in a dose-dependent manner. Furthermore, the CD40 binding protein was identified as a 39 kD protein that is selectively expressed on the membranes of activated, but not resting Tₕ. Both CD40-Ig and a mab specific to the 39 kD CD40 binding protein blocked B-cell activation by PM

Although a number of membrane proteins have been implicated in Tₕ-dependent B-cell signalling, evidence presented herein dismisses the contribution of some molecules (LFA-1, CD4, CD3, α,β TCR, ICAm-1) and implicates CD40 as the B-cell receptor for cognate signalling by Tₕ. Data show that CD40-Ig and a mab specific to the CD40 binding protein inhibits Th-dependent B-cell activation.

The ligand for CD40 is a 39 Kd protein that is expressed on activated, but not resting Tₕ. Biochemical studies indicate that the 39 kD protein is a single chain molecule since electrophoretic migration was not influenced by reducing agents. Based on the functional studies presented in this study, both activated Tₕ1 and Tₕ2 express the 39 kD CD40 binding protein. This is consistent with the functional studies that show both Tₕ1 and Tₕ2 induce B-cell cycle entry. In an attempt to further characterize the 39 kD protein, cDNA encoding CD proteins in the MW range of 39 kD (Cd 53, CD27 and CD69) were transiently transfected into COS cells and the cells were tested for CD40-Ig binding. None of the transfected COS cells expressed proteins that bound CD40-Ig. It is therefore suspected that the 39 kD protein is not one of these CD proteins.

The biochemical basis for signal transduction between Th and B-cells has been elusive. The identification of CD40 as the signal transducing molecule for T cell help focuses attention on specific biochemical pathways known to be coupled to the CD40 molecule. CD40 is a member of the nerve growth factor receptor (NGFR) family by virtue of the presence of four cysteine-rich motifs in its extracellular region. Signalling through CD40 by mab has been shown (Uckun et al., J. Biol. Chem. 266:1 7478-17485 (1991)) to involve the activation of tyrosine kinases resulting in the increased production of inositol triphosphate and the activation of at least four distinct serine/threonine kinases. Based on information obtained from signaling through other members of the NGF receptor family, it is anticipated that interaction between activated Tₕ and B will result in many of the same biochemical processes.

For immunofluorescence binding studies, CD40 Ig fusion protein was conjugated with biotin using biotin-succinimide (Sigma). Flow cytometry analysis was then performed by two-step staining using phycoerythrin (PE)-streptavidin (Becton-Dickinson) with a Coulter Epics C instrument. Representative results of screening multiple T cell lines is presented below. The Jurkat and HSB2 cell lines were found to bind specifically, whereas other T cell lines including CEM, HPBALL, and murine thyoma did not bind the CD40 Ig fusion protein (FIG. 7).

### EXAMPLE 2

### Use of Anti-gp39 Antibody for Treatment or Prevention of Lupus

Systemic lupus erythematosus (SLE) is a disease characterized by the production of a variety of pathogenic autoantibodies (Boumpas, Ann Int Med, 1995). These autoantibodies cause damage directly through recognition of epitopes on normal cells or indirectly through the formation of immune complexes which can deposit in normal tissue and activate the complement cascade.

As with normal antibody responses, it is now clear from studies of SLE and lupus-like disease in inbred strains of mice that lupus B cell autoantibody production depends upon cooperation from CD4+ T helper (Th) cells. An example comes from studies of the classic murine model of SLE, the female offspring of NZB/NZW mice, who are afflicted with a disease similar to human SLE in many respects, including the production of autoantibodies and the development of an immune complex glomerulonephritis. Treatment of these mice with depleting anti-CD4 antibodies prevents and actually reverses nephritis. Unfortunately from a therapeutic standpoint, even brief treatment trials of anti-CD4 antibody in human autoimmune disease may lead to prolonged depletion of this critical lymphocyte subset in some cases. Moreover, efficacy has been disappointing in the small clinical trials of anti-CD4 antibody in diseases such as rheumatoid arthritis.

More specific immunosuppression is now feasible due to the identification of several of the molecular interactions involved in Th cell help, making it possible to target only those Th cells actively involved in providing help for antibody production. One example is the second signal delivered by the interaction between CD28/CTLA4 on the Th cell and B7.1/B7.2 on the B cell. Blocking antibodies to either participant can render the T cell hyporesponsive in vitro. Recently, a fusion protein consisting of the extracellular domain of murine CTLA-4 linked to a murine Ig Cg2a chain was shown to block autoantibody production and prolong life when given to NZB/NZW mice, even with advanced stages of disease, presumably by competitively inhibiting the binding of B7.2 to endogenous CTLA-4 in treated mice.

Experimental Results.

We studied the effects of administration of anti-CD40L antibody from 4 to 10 months of age on murine SLE in NZB/NZW mice. Anti-CD40L antibody treatment significantly reduced anti-DNA autoantibody production and renal disease and prolonged survival in these mice, without eliciting an anti-antibody response or causing readily apparent impairment of the humoral immune system in treated mice. These data are contained in FIG. 9. At 11 months of age, 60% of anti-CD40L antibody-treated mice, but no untreated or HIg-treated mice, were alive (FIG. 9). Histologic examination of kidneys in long-term survivors after anti-CD40L antibody treatment revealed negligible pathology and the absence of significant immune deposition in glomeruli.

The data obtained also suggested that anti-CD40L antibody therapy may circumvent two major concerns with the use of anti-lymphocyte cell-surface molecule antibodies for immunotherapy: the development of an anti-antibody response and prolonged post-treatment immunosuppression. In our cohort, mice who responded to anti-CD40L antibody treatment in terms of survival at 11 months did not develop an anti-antibody response, an almost invariable phenomenon in animals receiving antibodies derived in another species. We postulate that this is due to the prevention by anti-CD40L antibody of the production of antibody to itself. In studies of normal mice treated with the same anti-CD40L antibody, MR-1, no anti-MR-1 responses were seen suggesting that the responses we have observed in the NZB/NZW mice may represent a particular hyperreactivity limited to autoimmune mice. In any case, this observation has important implications for the therapeutic application of an anti-human CD40L antibody, since this serious complication of antibody therapy may be obviated with anti-CD40L antibody therapy, particularly with a “humanized” version of the antibody. In our study, mice who did develop an anti-anti-CD40L antibody response fared no better than control mice in their anti-DNA antibody production or survival, suggesting that, for whatever reason, antibody production by this subset of mice was not prevented by anti-CD40L antibody. The development of anti-anti-CD40L antibodies may have led to rapid clearing of subsequently administered anti-CD40L antibody, and thus loss of therapeutic efficacy. In support of this notion, as in mice treated with nonspecific hamster IgG, deposition of hamster antibody was identified in the glomeruli of the subset of non-responding anti-CD40L antibody-treated mice and not in anti-CD40L antibody-responders. This observation is consistent with anti-anti-CD40L antibody immune complexes being formed and deposited in the kidney with continued administration of anti-CD40L antibody, perhaps aggravating, rather than preventing, glomerulonephritis.

Finally, the demonstration that with cessation of anti-CD40L antibody treatment, the ability to mount an anti-KLH antibody response returns after at most four months establishes that the immunosuppressive effects of the antibody on the humoral immune system are potentially reversible and therefore anti-CD40L antibody treatment may be suitable for human therapeutic applications.

In these experiments, the NZB/NZW Fl mice were treated with 200 ug of MR-1 intraperitoneally twice a week from age 4-10 months. Also, treatment has also been effected by a treatment protocol, increasing the dosage to 250 mg of MR-1 three times a week intraperitoneally from 4 to 10 months of age. This dose had been used in the collagen-induced model of rheumatoid arthritis and resulted in 100% survival in treated mice (Durie, Science, 1993). Using this protocol, the NZB/NZW mice now have 100% survival at month 11 (FIG. 10), whereas two control groups of mice had all expired by age 10 months.

### Treatment of Established Lupus Disease

While the prevention of SLE in the NZB/NXW F₁ mice with anti-gp39 antibody is of interest, it is not predictive of the utility of this antibody to be able to therapeutically intervene in active progressed disease states. In order to determine if an established disease could be reversed with antibody treatment, a cohort of mice were allowed to develop 2-3+ proteinuria (equivalent to 100 mg/day to 500 mg/day), determined by urine dipstick. At that point, mice were randomly assigned to continue no treatment or to receive MR-1 at 100 μg/day. The ten untreated mice were all dead by 10 months of age. By contrast, 5 of 10 MR-1 treated mice were alive at 11 months of age. Three of these mice were healthy and without proteinuria. Intriguingly, one of these mice nevertheless has high-titer anti-DNA antibody (Table 1). Two of the 5 survivors appear sick and have high titer anti-DNA antibodies and 4+ proteinuria.

Thus, treatment with MR-1 after the development of renal disease was found to reverse disease in some mice. The finding in one responding mouse that treatment with MR-1 reversed proteinuria, but not anti-DNA antibody production, further raises the possibility of another mechanism for the MR-1 effect other than abrogating T-dependent antibody production.

Also, it was shown that treatment of NZB/NZW mice with tumor necrosis factor (TNF-α) significantly delayed the development of nephritis (Jacob, Science, 1988; Gordon, 1989). Interestingly, the severe form of nephritis seen in NZB/NZW F1 mice is due in part to dominant gene(s) from the NZW parent that map to the H-2 complex. The NZW mice have reduced levels of TNF-α that correlates with a polymorphism in the TNF-α gene which is located within the H-2 complex. The NZB/NZW F1 mice also have significantly reduced levels of TNF-α. Furthermore, treatment of NZB/NZW F1 mice with anti-IL-10 antibody substantially delayed the onset of nephritis and prolonged survival. The beneficial effect appeared to be mediated by an IL-10-induced upregulation of TNF-a levels in the treated mice as co-treatment with anti-TNF-a antibodies abrogated the anti-IL-10 response.

In a preliminary experiment, we also examined the levels of TNF-α by ELISA in a mouse from each of 4 treatment groups pre- and post-MR-1 administration. The levels are shown in Table 2. Only the mouse that responded to MR-1 treatment in terms of resolution of proteinuria and anti-DNA antibody production demonstrated an increase in TNF-α from 2.16 pg/ml pre-treatment to 35.01 pg/ml post-treatment. These data suggest that the MR-1 effect may be at least in part mediated through an increase in TNF-α in NZB/NZW F1 mice.

Moreover, these results substantiate the utility of anti-gp39 antibodies and active fragments thereof for treating human lupus, i.e., systemic lupus erythematosus or drug-induced lupus. In particular, these antibodies can be used for the therapeutic intervention of active, ongoing lupus which is often characterized by nephritis. Such antibodies should inhibit the progression of the disease and potentially even reverse the disease process.

TABLE 1

| Age | | | | | |
|---|---|---|---|---|---|
| (Months) | | | | | |
| Post | MR1 | | | | |
| Treatment | Treat- | Post | | Re- | |
| At | ment | Treatment | Remission | treatment | Duration |
| Proteinuria | (Weeks) | Proteinuria | (Weeks) | With MR1 | (Weeks) |
| 7 | 13 | +4 | N/A | N/A | — |
| — | | | | | |
| 9 | 6 | 0 | 0 | NO | — |
| — | | | | | |
| 6 | 6 | 0 | 12 | NO | — |
| — | | | | | |
| 5 | 6 | 0 | 11 | YES | 6 (0) |
| 0 | | | | | |
| 8 | 1 | DEAD | — | — | — |
| — | | | | | |
| 6 | 18 | DEAD | — | — | — |
| — | | | | | |
| 6 | 6 | 0 | 9 | YES | 3 (+4) |
| +4 | | | | | |
| 6 | 6 | DEAD | — | — | — |
| — | | | | | |
| 7 | 5 | DEAD | — | — | — |
| 6 | 2 | DEAD | — | — | — |

TABLE 2

| Mouse* | Age sample taken (mos.) | TNF-a (pg/ml)# |
|---|---|---|
| No treatment | 2 | 5.5 |
| | 9 | 3.1 |
| MR-1 Alive | 2 | 2.2 |
| | 9 | 35.0 |
| MR-1 Died | 2 | 0.0 |
| | 8 | 4.3 |

*one mouse from each treatment cohort.
#Values represent means of duplicates from assay using TNF-α ELISA kit from Genzyme, Cambridge, MA.

Various publications cited herein are hereby incorporated by reference in their entirety.

What is claimed is:

1. A method of treating lupus in a subject in need of such treatment which comprises administering a therapeutically effective amount of an anti-gp39 antibody or a fragment thereof that specifically binds gp39.

2. The method of claim 1, wherein said lupus is systemic lupus erythematosus.

3. The method of claim 1, wherein said lupus is drug-induced lupus.

4. The method of claim 1, wherein said antibody is a humanized anti-human gp39 antibody.

5. The method of claim 1, wherein said antibody is a chimeric anti-human gp39 antibody.

6. The method of claim 1, wherein said antibody is administered systemically.

7. The method of claim 6, wherein said antibody is administered to a lupus subject having lupus-associated renal disease.

8. The method of claim 7, wherein said administration results in reduction and/or reversal of said renal disease as evidenced by reduced proteinuria after anti-gp39 treatment.

9. The method of claim 1, wherein said anti-gp39 antibody is administered in combination with tumor necrosis factor.

10. The method of claim 1, wherein the dosage of anti-gp39 antibody ranges from 10 μg/ml to 1000 μg/ml.
